(19) United States
(12) Patent Application Publication
(10) Pub. No.: US 2023/0240524 A1
(43) Pub. Date: Aug. 3, 2023

(54) COLONOSCOPY SYSTEM

(71) Applicant: Jiawei Xu, Toronto (CA)

(72) Inventor: Jiawei Xu, Toronto (CA)

(21) Appl. No.: 17/649,451

(22) Filed: Jan. 31, 2022

(51) Int. Cl. A61B 1/31; A61B 1/015; A61B 1/04; A61B 1/018; A61B 1/00

(52) U.S. Cl. CPC A61B 1/31; A61B 1/04; A61B 1/015; A61B 1/018; A61B 1/00052

(57) ABSTRACT

A colonoscopy system that is configured to assist a user in execution of a colonoscopy. The present invention includes a body having a cylindrical center portion wherein the cylindrical center portion consists of a telescopic assembly. Located on opposing ends of the center portion are outer air members wherein the outer air members are operable to be inflated or deflated. Proximate the front end of the body is an image data collection member having a transparent capsule and a communications module communicably coupled to an external computer network. The body includes an outer wall having a plurality of outer air passages and outer water passages. A piston assembly is operably coupled to the rear end of the body and includes a plurality of pistons configured to facilitate introduction of pressurized water and air into the body. The colonoscopy system further is operably coupled to a direct display.

### FIELD OF THE INVENTION

The present invention relates generally to healthcare apparatus, more specifically but not by way of limitation, a colonoscopy system wherein the present includes elements such as a new manner of movement via water and air and wherein data collected is further interpreted with artificial intelligence.

### BACKGROUND

Colorectal cancer is one of the most common malignant tumors, and its incidence is the highest in the world. Incidences of colorectal cancer mostly occur in the digestive system with a malignant tumor being the most prevalent. In 2018, the colorectal cancer cases exceeded over five hundred million with the death rate for late detection patients exceeding fifty percent. The current five-year colorectal survival rate in countries such as China is less than 40%, which is much lower than that of the United States, Japan, and South Korea. The survival rate in countries such as China is low due to more than eighty five percent of the patient detection occurring too late. Alternatively, early colorectal cancer improves the five-year survival rate, which can exceed 95% after treatment is implemented. One issue in some countries such as but not limited to China is early detection and diagnosis is less than ten percent, which is significantly lower than that of Japan and South Korea country. In developed countries in Europe and the United States, a relatively mature national colorectal cancer screening system has been established. In contrast, colorectal cancer screening in countries such as but not limited to China began in the 1970 but only certain districts and cities actually have colorectal screening. Compared with European and American countries, the main factors restricting early colon cancer screening in China at this stage are numerous. One issue is that China has a large population, which creates cost issues to execute effective screening. Another issue is that there are huge differences in the level of medical care in different regions across various countries. Colonoscopy is a difficult and not popular procedure however the risk of not executing this procedure for the patient is high as colorectal cancer has no obvious signs with early symptoms being mild and easily confused with other illnesses.

The incidence rate of colorectal cancer in China is 23.7 per 100,000 people and the incidence rate increases with age. The incidence rate is low before the age of 50 but after the age of 50 the incidence tends to increase rapidly. Most developed regions such as Europe and the United States use between the ages of 50 and 75 a colonoscopy procedure as a screening test for colorectal cancer. The United States has implemented a lower minimum age for screening that is 45 years old. In countries such as but not limited to China, the correlation between the incidence rate and age shows that the incidence rate increases at the age of 40 years old, while the acceleration is even more pronounced over the age of 50. The gap between urban and rural areas in China for healthcare is significant with rural areas having a reduced availability of facilities. Developed affluent areas have more doctors, colonoscopes and other examination equipment. The relatively poor and remote areas lack the equivalent conditions, whether it is the doctor’s ability or the equipment design. Causes of diseases related to the digestive system are factors such as but not limited to eating habits that include foods higher is nitrates, alcohol and saturated fat. The difference in eating habits caused by local differences is preserved in tradition and culture, does not change. This leads to a high incidence of digestive system-related cancers in the local area. Many countries such as China, Japan and the US have an aging trend that is increasing. There is a huge gap between the needs of medical practitioners and the actual doctors who can provide diagnosis and treatment.

Accordingly, there is a need for a response to the dilemma described in the background wherein a new type of colonoscopy integrated system addresses the existing defects of traditional colonoscopies. The present invention includes a manual operation state that is the same as that of the traditional colon colonoscopy but further provides fully automatic and semi-automatic lens entry modes. User of the present invention can switch between the above three modes so as to solve various difficulties encountered in colonoscopy procedures.

### SUMMARY OF THE INVENTION

It is the object of the present invention to provide a colonoscopy system configured to provide execution of a colonoscopy wherein the present invention includes a body that is generally cylindrical in shape.

Another object of the present invention is to provide a colonoscopy system that is operable in three modes wherein the body of the present invention includes a center section wherein the center section is telescopic.

A further object of the present invention is to provide a colonoscopy system configured to provide execution of a colonoscopy wherein the body of the present invention includes an expandable rear member.

Still another object of the present invention is to provide a colonoscopy system that is operable in three modes wherein the body of the present invention wherein the body includes a expandable front member wherein the expansion of the rear member and front member are pneumatically controlled.

An additional object of the present invention is to provide a colonoscopy system configured to provide execution of a colonoscopy wherein the body further includes internal channels for distribution of air and water.

Yet a further object of the present invention is to provide a colonoscopy system that is operable in three modes that further includes a plurality of piston-roller assemblies.

Another object of the present invention is to provide a colonoscopy system configured to provide execution of a colonoscopy that includes an image data acquisition member.

Still an additional object of the present invention is to provide a colonoscopy system that is operable in three modes wherein the body further includes integrally as a part thereof a wireless communication module.

Yet another object of the present invention is to provide a colonoscopy system configured to provide execution of a colonoscopy that further includes a piston control device.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a cross section of a portion of the piston assembly; and

FIG. 2 is a perspective detailed view of a section of the piston assembly; and

FIG. 3 is a cross-sectional end view of the body of the present invention;

FIG. 4 is a side view of the body of the present invention with the telescopic portion extended; and

FIG. 5 is a perspective view of the body of the present invention; and

FIG. 6 is a perspective view of the present invention completely assembled; and

FIG. 7 is a schematic layout of the data system and portions thereof operably coupled to the present invention.

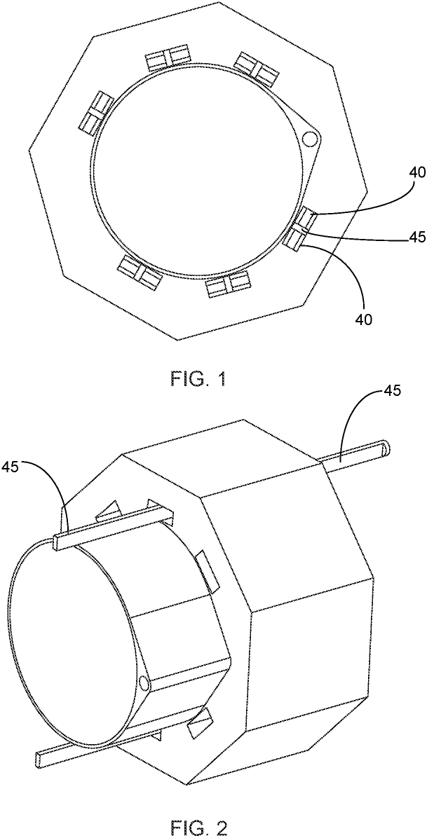

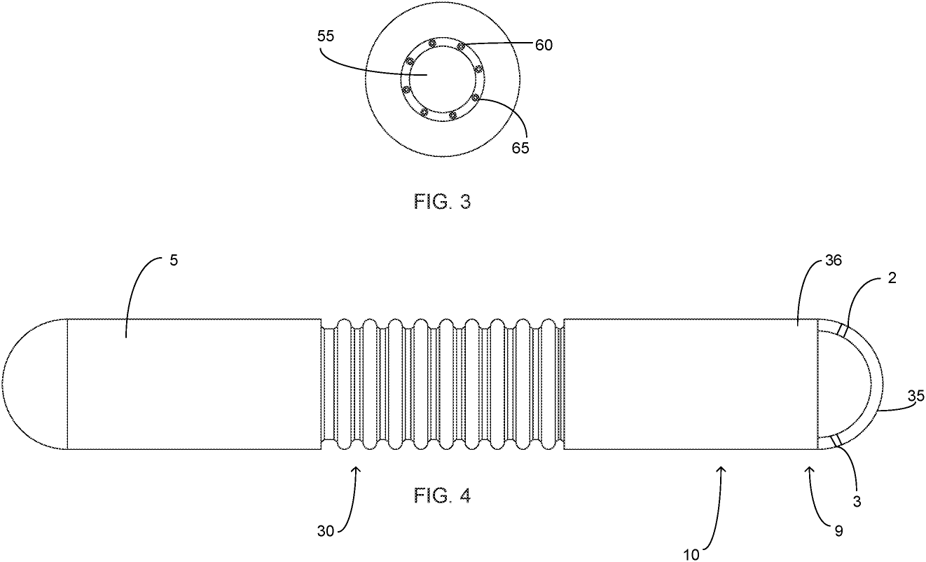

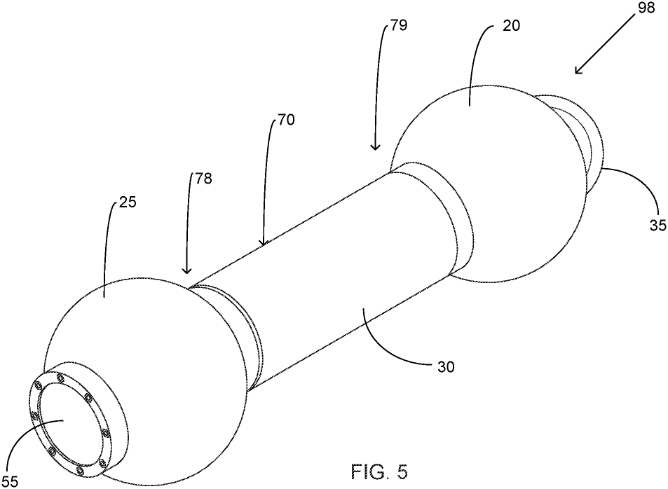

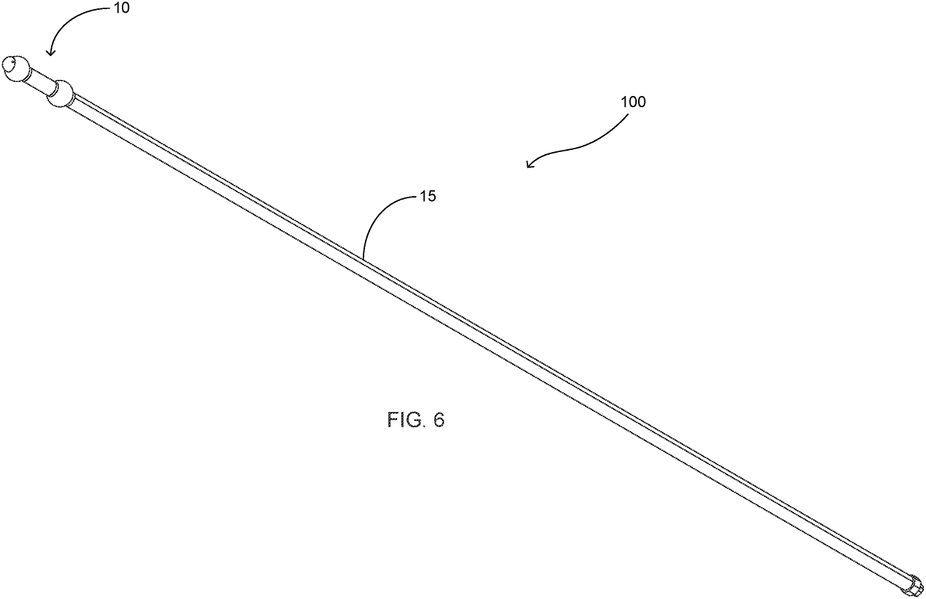

### DETAILED DESCRIPTION

Referring now to the drawings submitted herewith, wherein various elements depicted therein are not necessarily drawn to scale and wherein through the views and figures like elements are referenced with identical reference numerals, there is illustrated a colonoscopy system 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted as a part hereof, the colonoscopy system 100 includes conventional data system components(not particularly illustrated herein) that are utilized during the operation of the colonoscopy system 100. This includes networked data systems such as a hospital data system, a central data system and a supporting display system that would include direct display system. It should be understood within the scope of the present invention that various combinations of data systems could be operably coupled to the colonoscopy system 100 and an exchange of data such as but not limited to image data and control signals occur through the networked components. The body 10 of the colonoscope is controlled by the piston assembly 15. The body 10 is releasably secured to the piston assembly 15 utilizing suitable techniques. The body 10 includes outer air members 20, 25 having a hydrodynamic telescopic assembly 30 intermediate thereto. The body 10 further includes an image data collection member 35 wherein the integrated capsule 36 and forms a closed environment. The piston assembly 15 is operably controlled by a conventional fly-by-wire controller that is configured to provide operation of the body 10 as is further described herein. As will be further discussed herein air and water flow into the outer members 20, 25 and hydrodynamic telescopic assembly 30 to facilitate the inflation and deflation of the outer air members 20, 25 as well as expansion and contraction of the telescopic assembly 30. The outer air members 20, 25 being located on opposing ends of the telescopic structure 30 foot have an appearance similar to that of a dumbbell subsequent the outer air members 20, 25 being inflated. When deflated the shape is rod-like and the diameter of the outer air members 20, 25 are slightly smaller than the smallest diameter of the colon. The maximum bending of the telescopic assembly can be greater than 90 degrees and the maximum telescopic extension is at least is greater than six centimeters.

During a colonoscopy procedure, the body 10 of the is operably coupled to the piston assembly 15 which includes all of the necessary electric, pneumatic and water inputs so as to provide control of the body 10. The piston assembly 15 provides control of the outer air members 20, 25 and telescopic assembly 30 so as to provide operational movements such as but not limited to forwarding, turning, pitching, positioning, and opening the passage. The body 10 is operably coupled to the piston assembly 15 which has the necessary electronic connections to provide operational control of the body 10. The body 10 includes releasably secured telescopic assembly 30, which further includes the detachable outer air members 20, 25. Proximate the first end 9 of the body 10 is the image data collection member 35. Opposedly located thereto is the power supply 5. In a preferred embodiment the tip 98 can be detached from the outer air members 20, 25 and the hydrodynamic telescopic assembly 30 and manufactured from disposable medical plastic consumables. The compact components and the close integration of the three enable the integration of the internal image data acquisition and power supply 5. The piston assembly 15 is located at the rear end is always outside the body during a colonoscopy procedure, and as such is not contaminated by the intestinal tract. Subsequent execution of the traditional colonoscopy the colonoscopy system 100 needs to be cleaned as a whole and the process of disinfection is transformed into a process of discarding medical consumables, in this instance the body 10 and reinstalling. Furthermore, the piston assembly 15 is controlled separately by pistons 45 that direct inflation and deflation of the outer air members 20, 25 as well as provide control of the and hydrodynamic telescopic assembly 30.

The pistons 45 are used to control the telescopic assembly 30 in the four directions of up, down, left, and right. The specific principle of the movement control is as follows. When the piston 45 is driven by the rollers 40 into body 10, air is forced into the outer air members 20, 25. Alternatively, when the rollers 40 reverse the pistons 45 in the opposing direction the air introduced into the outer air members 20, 25 is pumped out. The operation of the hydrodynamic telescopic assembly 30 is consistent with the inflating and deflating operation of the outer air members 20, 25. The execution of bending of the telescopic assembly 30 is as follows. Due to the material construction of the hydrodynamic telescopic assembly 30, it is under pressure and as such the axial change is much greater than the tangential change. When one side of the telescopic assembly 30 is pressurized with water, the opposing side is decompressed or unchanged. At this time, the whole structure will bend to the side having the least amount of pressure. An omnidirectional angle greater than ninety degrees can be achieved. Through utilization of the controls integral with the piston assembly 15 the aforementioned movements can be realized in the intestine along the intestinal tract.

The body 10 will execute bending movements so as to avoid having to straighten the intestines. The body 10 includes a passage 55 in the middle thereof for water removal during the colonoscopy procedure. A small outer water passage 60 extends through the body 10 and is operably coupled to a pressurized water source and is configured to utilize water pressure as required during the colonoscopy procedure for tasks such as but not limited to cleaning the field of vision. The body 10 further includes an outer air passage 65 wherein the outer air passage 65 is operable to deliver pressurized air so as to assist in execution of the immediately aforementioned tasks. Both the air and water exit aperture 3 during procedure. A plurality of outer air passages 65 and outer water passages 60 are present so as to be utilized to clean the field of vision as needed during colonoscopy. The ends 78, 79 of the detachable tube body 70 are releasably coupled to the outer air members 20, 25. There is a biopsy forceps exit aperture 2 through which the biopsy forceps can take biopsy operations. The biopsy forceps exit aperture 2 is operably coupled to the outer water passages 60 and outer air passages 65.

The body 10 part consists of a outer air members 20, 25 and hydrodynamic telescopic assembly 30 and the image data collection member 35 as well as the and power supply 5 forming an integrated capsule. The body 10 is manufactured from a flexible, soft material. The water pressure is controlled by the pistons 45, wherein the pressurized water is directed into the outer water passages in order to achieve expansion and contraction of the telescopic assembly 30. An outer layer at both ends is a thin film and the inner layer is a hard tube wall water passage 55 inside the tube wall. The tip 98 is made of semi-spherical transparent material. Portion 36 adjacent the tip 98, image data collection member 35 includes necessary electronics to facilitate communication and image acquisition.

The power supply 5 provides the necessary power to complete the colonoscopy procedure. During colonoscopy procedures the image data collection member 35 is operably coupled to a communications network that is connected with the hospital-side data system and the visual data captured by the image data collection member 35 is displayed in real time through a direct display system available to the user of the colonoscopy system 10. The display system part is divided into the direct display system and the processed video display system as described above. The direct display system mainly serves the on-site operation of doctors, including real-time display of direct collection.

Images and amounts of artificial intelligence are provided to assist in annotating information. The images are displayed on a touch screen display device, wherein the body 10 is manipulated by the touch screen to perform real-time image operations such as zooming, freezing, and grabbing. After processing, the video display system can be divided according to different users in order to the results for three types of data. First, the patient’s personal multiple colonoscopy which include data results of the inspection and data results of previous procedures in order to show the changing trends in the time dimension. Additionally, the data can be compared to other individuals in the same demographic and age group. Lastly, the data can be presented to the user of the colonoscopy system 100 segmented by regions of the colon.

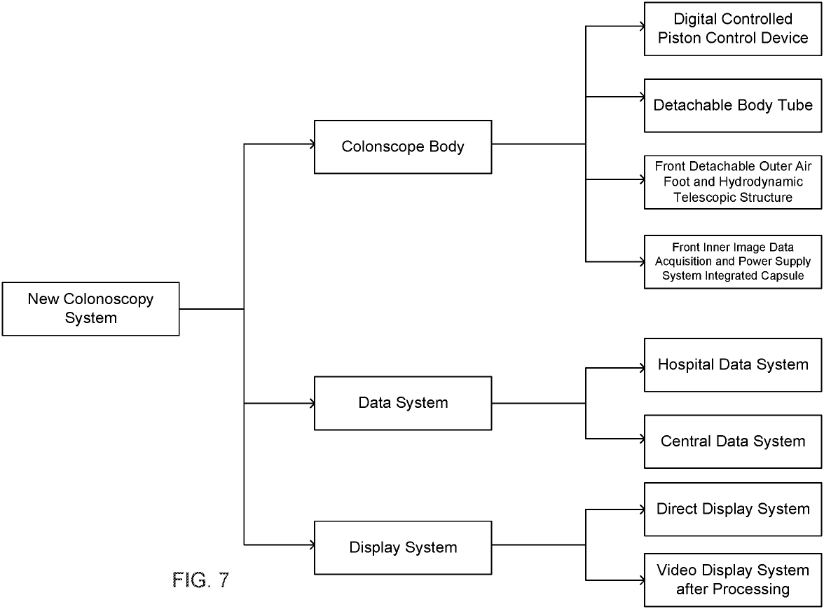

The data system portion of the present invention(as represented herein in FIG. 7) is divided into hospital-end data system and central data system. The hospital-end data system provides data support for the direct display system while providing data to the central data system. The central data system will pass the preliminary screening and send the data of patients with suspected lesions to the clinic. For hospitals with interruption capability, the doctors and artificial intelligence-assisted diagnosis technology are used for the second confirmation, and the final diagnosis result is generated. Final results are sent to the original data source hospital and the examinee. In order to enable remote diagnosis, the central data system uses the collected data to form a regional screening archive, the inspector’s lifetime database based on the inspected, and the hospital is a database of hospital inspection, diagnosis and treatment of the unit. Ultimately, the colonoscopy system 100 provides a life cycle of data for the inspected that includes traceability to the local region. This data can be provided to local healthcare practitioners to study the local intestinal disease incidence and prevention status for the region statistical data and related suggestions. Additionally, this data can be utilized to provide examination, diagnosis and treatment for the digestive system as well as the storage of medicines and the development of operations provide support.

The operation process of the colonoscopy system 100 is divided into three modes: manual, fully automatic and semi-automatic. In manual mode the outer air members 20, 25 are fully deflated and the hydrodynamic telescopic assembly 30 is in a non-extended position. The user will push the body 10 from the outside into the intestine and the data transmission of the colonoscopy is the executed. The fully automatic mode is used in the case of obstacles to manual lens entry. In this mode, the back-end fly-by-wire piston assembly 15 controls the body 10 having the outer air members 20, 25 and hydrodynamic telescopic assembly 30 drives the body 10 into the intestine via water pressure to extend the telescopic assembly 30 and inflation/deflation of the outer air members 20, 25 as described herein. Implementing this mode will facilitate passing through a blocked area at a steady rate. The semi-automatic mode is used in situations where suspected lesions are found. Manual control of the piston assembly 15 so to control the detachable outer air members 20, 25.

With the movement of the hydrodynamic telescopic assembly 30, it is a collection of the inner image data collection and power supply system of the tip. The user of the colonoscopy system 100 can determine whether they need to implement a biopsy procedure. The biopsy procedure is performed by removing the biopsy forceps from the body 10 and inserting into body so as to exit from the biopsy exit aperture 3. During a biopsy operation, biopsy forceps, lesions are all located in the field of vision of the image data collection member 35 and are on the direct display to guide the operation. The user can use the real-time screen displayed by the direct display system to determine whether or not to utilize water or air to cleanse the area for improved visibility and further to identify when to pump the water away.

The water passage 55 is configured to facilitate removal of water that is injected proximate to the body 10 via outer water passages 60. Controlling an external pump air supply, water supply and water evacuation are executed via utilization of an external pump. During colonoscopy, the collected data is not only uploaded to the direct display system but also uploaded to the hospital data system. Subsequent data processing, data is uploaded to a central data system. Artificial intelligence is subsequently utilized to identify any lesions or similar growths for further analysis.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A colonoscopy system comprising:
a body, said body having a first end and a second end, said body being cylindrical in shape having a wall, said body having a telescopic assembly, said telescopic assembly comprising a center section of said body, said body further having a hollow passage therein;
a first outer air member, said first outer air member being releasably secured to said first end of said body, said first outer air member operable to be inflated or deflated during a colonoscopy procedure;
a second outer air member, said second outer air member being releasably secured to said second end of said body, said second outer air member operable to be inflated or deflated during a colonoscopy procedure;
an image data collection member, said image data collection member being proximate said second end of said body, said image data collection member configured to collect photographic images, said image collection data member having electronics configured to transmit collect data to at least one source via a network;
a piston assembly, said piston assembly being releasably secured to said second end of said body, said piston assembly being operably coupled to said body so as to provide operation thereof, said piston assembly including a plurality of pistons configured to supply air and pressurized water to said body.

2. The colonoscopy system as recited in claim 1, and further including at least one outer air passage, said at least one outer air passage being formed in said wall of said body, said at least one outer air passage configured to distribute air to proximate the image data collection member.

3. The colonoscopy system as recited in claim 2, and further including at least one outer water passage, said at least one outer water passage being configured to deliver pressurized water to said telescopic assembly and to an area of a colon proximate said image data collection member.

4. The colonoscopy system as recited in claim 3, wherein upon deliver of pressurized water to said telescopic assembly said telescopic assembly extends in length.

5. The colonoscopy system as recited in claim 4, wherein said body is configured to be manipulated by said piston assembly in four opposing directions.

6. The colonoscopy system as recited in claim 5, wherein said piston assembly includes rollers that are operably coupled to said plurality of pistons so as to facilitate movement of said plurality of pistons.

7. The colonoscopy system as recited in claim 6, wherein said body further includes a power supply proximate said first end.

8. The colonoscopy system as recited in claim 7, wherein said telescopic assembly is moved in a bending motion through control of a water pressure being directed thereto.

9. The colonoscopy system as recited in claim 8, and further including a biopsy forceps aperture, said biopsy forceps aperture being proximate said first end, said biopsy forceps aperture operable to permit a forceps to extend outward therefrom.

10. The colonoscopy system as recited in claim 9, and further including a water aperture, said water aperture operably coupled to said at least one outer water passage, said water aperture being proximate said image data collection member so as to clean an area proximate thereto.

11. The colonoscopy system as recited in claim 10, wherein the image data collection member is communicably coupled to an external computer network and a direct display wherein the direct display is proximate the colonoscopy system.

12. The colonoscopy system as recited in claim 11, wherein the colonoscopy system includes a manual mode, a fully automated mode and a semi-automatic mode.

13. The colonoscopy system as recited in claim 12, wherein image data presented on the direct display is divided into three categories.
